(19) United States
(12) Patent Application Publication
(10) Pub. No.: US 2002/0012651 A1
(43) Pub. Date: Jan. 31, 2002

(54) Release of therapeutic agents in a vessel or tissue

(76) Inventor: Marvin P. Loeb, Huntington Beach, CA (US)

Correspondence Address: OLSON & HIERL, LTD., 36th Floor, 20 North Wacker Drive, Chicago, IL, 60606, US

(21) Appl. No.: 09/852,528

(22) Filed: May 10, 2001

Related U.S. Application Data
(60) Provisional application No. 60/202,877, filed on May 10, 2000.

(51) Int. Cl. A61K 48/00; A61K 38/19; A61K 39/395; A61K 38/43; A61K 9/127

(52) U.S. Cl. 424/85.1; 424/450; 424/130.1; 514/44; 424/94.1

(57) ABSTRACT

Particles larger than 7 microns in diameter and containing a therapeutic material are described. Such particles are sufficiently large to lodge in tissue or a blood vessel and can be made to rapidly degrade therein before creating infarcts. Rapid degradation of liposomes containing a therapeutic agent can be achieved by either preparing the liposome in a high salt solution or incorporating in the liposome a component which is stable at or below room temperature, but which becomes unstable at 35° C. or higher. Such particles are useful in delivering, for example, angiogenic growth factors or genes coding for angiogenic growth factors into the coronary arteries of the heart or the heart muscle, as well as other therapeutic agents into organs or tissues or blood vessels feeding the same to achieve a desired effect.

### FIELD OF INVENTION

This invention relates to the delivery of therapeutic agents to a patient.

### BACKGROUND OF THE INVENTION

Therapeutic agents, including inorganic and organic chemical entities, e.g., antineoplastic agents, antibiotics, and the like; biologically active substances, such as human genes, proteins, enzymes, antibodies, and the like, as well as genetically engineered copies of the same; nutrients, vitamins, minerals, and other plant and animal substances; and other therapeutic or medicinal compositions are referred to herein collectively as Therapeutics.

Therapeutics which are not adversely affected by the digestive system may be administered orally. Therapeutics which are adversely affected by the digestive system may be encapsulated or coated with an enteric material able to resist the acids of the stomach, infused into an artery or vein for general systemic distribution throughout the body or injected intramuscularly. Toxic Therapeutics are often infused into a major artery through a percutaneously inserted catheter, in order to dilute them in a relatively large volume of blood. Sometimes, Therapeutics are injected into a tissue or organ for a localized effect. Blood flow through the tissue or organ, however, usually carries away most of the Therapeutic into the general circulation, before it has had time to exert its intended effect in the target organ or tissue. Even if the Therapeutic is designed to bind to receptors unique to the tissue or organ, or if the Therapeutic is attached to a carrier to enhance its absorption, much of the Therapeutic still passes through the target tissue or organ too quickly to be absorbed and is dispersed in the general circulation, where it can cause adverse effects.

It would be desirable to limit the action of a Therapeutic to a particular tissue or organ. Since blood flows rapidly through the capillaries of an organ or tissue into the general circulation, it would be desirable to cause the Therapeutic to pass through large arteries, but not through small arteries and capillaries, exerting its effect in the target tissue or organ. Presently, a Therapeutic, whose effect is desired in the tissue of the heart muscle, for example, passes through the arteries, capillaries, and veins of the heart wall in less than one second and enters the general circulation before it can fully exert its intended effect on the heart tissue.

While injection into an artery or vein close to the skin or into a muscle can be accomplished relatively easily, injection into the brain, heart, lungs, kidneys, liver, pancreas or other internal organ is a substantially more demanding and dangerous procedure. Consequently, it would be desirable if the Therapeutic did not quickly pass through the tissue or organ into the general circulation, before it is able to accomplish its Therapeutic effect therein.

Recently, the infusion into a coronary artery of an angiogenic growth factor, or the genes that cause cells to express (manufacture) angiogenic growth factors, through a percutaneously delivered catheter, such as is used to deliver a radio-opaque material for an angiogram, has been proposed to create new blood vessels in the heart muscle. However, the time required for absorption by cells of a protein such as fibroblast growth factor (FGF) or vascular endothelial growth factor (VEGF) generally exceeds one minute, far longer than the residence time of the growth factor in the arteries, capillaries and veins of the heart muscle. As a result, a clinical trial in which VEGF-165 was infused into a coronary artery, through such a catheter, produced no better result than a placebo (saline). See, for example, Henry et al., Circulation, 100 (18):Abstract No. 2509 (Nov. 2, 1999).

Since absorption of naked DNA by cells takes almost one minute, and since an enzyme in the blood decomposes DNA in approximately 30 seconds, a mechanism designed to prevent unintended uptake of DNA by cells, the injection or infusion into a coronary artery of naked DNA, for example, to cause the expression of an FGF or VEGF in the heart, suffers from the same disadvantage. Even when naked DNA was injected into a leg muscle of persons suffering from peripheral atherosclerosis, who were at the risk of amputation due to gangrene, a substantial percentage of the recipients received no benefit. See, for example, Baumgartner, I. et al., “Constitutive Expression of phVEGF₁₆₅ After Intramuscular Gene Transfer Promotes Collateral Vessel Development in Patients with Critical Limb Ischemia”, Circulation 97:1114-1123 (1998).

Inserting the gene for an angiogenic agent into the plasmid of a virus, which has the ability to rapidly invade cells, shortens the absorption time to about 20 seconds, still too long for use in beating heart. Furthermore, when an adenovirus, into whose plasmid an angiogenic gene was inserted, was infused into a coronary artery, in addition to the virus being later detected in the brain, lungs, liver, kidneys and testes, where the angiogenic agent could help a tumor extend its blood vessel system, the immune system attacked and destroyed the affected cells in a few weeks, aborting the desired expression of the angiogenic agent. See Berlener, “Development of Adenovirus Vectors for the Expression of Heterologous Genes”, Biotechniques 6:616-629 (1988) and Barr et. al., “Efficient Catheter-Mediated Gene Transfer into the Heart Using Replication-Defective Adenovirus,” Gene Therapy 1:51-58 (1994). Encapsulation of Therapeutics in very small liposomes has been proposed. Such liposomes are absorbed by cells without prompting an immune response. However, liposomes produced in accordance with U.S. Pat. Nos. 4,089,801, 4,229,360, 4,235,871, 5,017,359 are less than 2 microns in diameter (typically 50-350 nanometers). Such liposomes pass easily through the capillaries into the general circulation, distributing their contents throughout the body, where they can produce undesirable effects.

An objective of the invention is to cause a Therapeutic to lodge in tissue or in arterioles and capillaries for a time period sufficient to permit a Therapeutic to be released, and to exert its effect therein, without causing infarcts (blockages) that might cause damage to the tissue supplied by these vessels. Multiple infarcts in small vessels and infarcts in larger vessels, if they persist for more than a very few minutes, could be deleterious if such were to occur, particularly in the heart, brain, lungs and other organs.

The present invention solves these problems and permits Therapeutics to remain in the target tissue or organ for a sufficient time to be absorbed and exert their therapeutic effect on the cells therein, without causing long lasting infarcts or adverse effects in other tissues.

### SUMMARY OF THE INVENTION

A Therapeutic is incorporated into physiologically compatible, rapidly biologically degradable or dissolvable particles by means known in the art. These particles have a diameter greater than 7 microns, preferably 10 to 60 microns or larger. Particles of such size will not pass through the capillaries of a tissue or organ. While some will lodge in arterioles (small arteries), all will pass through larger arteries without causing an infarct. When a degradable or dissolvable particle containing a Therapeutic lodges in an arteriole or capillary, often at a bifurcation of the vessel into two smaller vessels, the surface of the particle facing the direction of blood flow will degrade or dissolve first, due to the blood pressure exerted thereon. This generally occurs before the remainder of the particle blocking the vessel is degraded or dissolved, and results in a substantial portion of the Therapeutic being released into the vessel and, hence, into the target tissue for a period of time, depending on the speed of degradation or dissolution of the particle. Thus, residence time for the Therapeutic in the patient's body fluid, e.g., blood stream or plasma, about one-half to several minutes, preferably about 1 to 2 minutes and not longer than about 3 minutes, can be obtained by formulating the particle or vesicle to rapidly degrade or dissolve within such period of time.

More particularly, a physiologically compatible particle embodying the present invention contains therewithin a therapeutic agent, has a diameter of at least about 7 microns but no more than about 300 microns, and exhibits a residence time in a body fluid, e.g., blood or plasma, of at least one-half minute but no more than about 3 minutes. The physiologically compatible particle can be a liposome, a microparticle, and the like. A method aspect of the present invention provides a convenient approach for introducing a therapeutic agent into the vasculature of a patient by injecting into the patient's blood stream physiologically compatible, discrete particles that contain the therapeutic agent, have a particle diameter of at least about 7 microns but no more than about 300 microns, and have a residence time in the patient's blood stream of at least about one-half minute but no more than about 3 minutes.

The therapeutic agent that can be administered in the foregoing manner can be hydrophilic, lipophilic, or a moiety that has a hydrophilic portion as well as a lipophilic portion. Any given particle can contain one or more of the aforementioned types of therapeutic agents. The therapeutic agents themselves can be selected from a wide variety of medicaments such as biologically active substances, nutrients, vitamins, organic compounds, inorganic compounds, e.g., chemotherapeutic agents such as antibiotics, cisplatin, carboplatin, and the like. Particularly preferred biologically active substances are filtered bone marrow, i.e., autologous or embryonic bone marrow, stem cells, i.e., autologous or embryonic stem cells, proteins, enzymes, monoclonal antibodies, fibroblast growth factor, vascular endothelial growth factor, cytokines, and the like.

Liposomes can be created with a diameter of 7 microns or larger, preferably 10 to 20 microns in diameter or larger, in which a Therapeutic can be incorporated. Liposomes with a diameter of 10 or more microns are sometimes called “giant” liposomes or vesicles.

Liposomes can be created with a single lamellar layer, surrounding an aqueous core. Multi-lamellar liposomes can be created with an aqueous core, surrounded by multiple concentric layers of lipids, each lipid layer being separated by an aqueous layer. A hydrophilic Therapeutic can be trapped in the aqueous core, as well as in the aqueous layers between the lipid lainellar layers of multi-lamellar liposomes. A lipophilic Therapeutic can be trapped in the lipid layer(s) of liposomes.

If liposomes with a diameter of 7 microns or larger containing a Therapeutic are formed in a high salt solution and injected into tissue or a blood vessel, water will flow into the liposome to bring the osmilality of the aqueous content of the liposome down to that of blood, the liposome will bloat and burst in less than a minute or two, releasing the Therapeutic in the tissue or the vessel in which the liposome is lodged.

Alternatively, a Therapeutic can be incorporated into a liposome with a diameter of 7 microns or larger of which at least one component is stable at room temperature, but which becomes unstable or degrades at 35° C. or higher temperatures, causing the liposome to degrade and release the Therapeutic in the target tissue or vessel where the liposome is lodged when its temperature exceeds 35° C.

If injected into a blood vessel, the larger the particle or vesicle, the higher up in the arterial system the Therapeutic will lodge. Particles or vesicles with a diameter greater than 300 microns could cause a blockage in a tributary of a coronary artery, which could create temporary ischemia and pain, even if degradation or dissolution occurs in less than 3 minutes. As a result, the use of particles larger than 300 microns in diameter should be judiciously applied.

If the Therapeutic is genes, they can be incorporated into liposomes or particles of a desired size. They can also be inserted into the plasmids of a virus, by means known in the art, and then incorporated into a liposome or bound in a particle of a desired size. When the liposome or particle degrades or dissolves, the genes are absorbed by cells, or the virus may penetrate cells, carrying the Therapeutic along.

If particles of more than one size are employed, they will lodge in both larger and smaller vessels, distributing their contents more widely throughout the vessel and the tissue it serves. To avoid causing infarcts in the heart, brain, lungs or other organ, the particles of vesicles should degrade, decompose or dissolve in a short period of time, from about 30 seconds to a few minutes, preferably about 1-2 minutes, but not longer than about 3 minutes. Utilizing combinations of two or more types of particles or vesicles, each with a different degradation or dissolution time, will enable very rapidly degrading (30 seconds to 1 minute) particles or vesicles or vesicles to be combined with less rapidly degrading (1 to 2 minute) particles or vesicles, preventing ischemia and pain from an excessive number of blockages, albeit temporary, occurring in the blood vessels of an organ or tissue at a given time.

Optimum distribution and release can be obtained by combining different sizes of particles or vesicles, with some degrading or dissolving in different time periods.

Stem cells are sufficiently large to lodge in very small arteries or capillaries. Such stem cells can be encapsulated in a liposome or incorporated into a biocompatible particle, which is designed to rapidly degrade or dissolve, and which has a diameter greater than the stem cells' normal size, causing them to lodge in relatively larger arteries of a tissue or organ. Filtered, physiologically compatible, autologous or embryonic bone marrow, which contains angiogenic agents, stem cells and other components can likewise be encapsulated in rapidly dissolving or degrading biocompatible particles or vesicles, and can be enriched, if desired, with stem cells isolated from peripheral blood, umbilical cord blood or an embryo, to increase the number of stem cells delivered to the organ or tissue.

### BRIEF DESCRIPTION OF THE DRAWINGS

In the drawings,

FIG. 1 is a schematic view of a particle, containing a Therapeutic, which has lodged at the bifurcation of a vessel into two smaller vessels.

FIG. 2 is a schematic view of the particle of FIG. 1, in which the surface facing the direction of blood flow is partially degraded or dissolved, releasing the Therapeutic into the blood vessel, while the remainder of the particle continues to block blood flow into the smaller vessels.

FIG. 3 is a schematic view of relatively smaller particles containing a Therapeutic, which have lodged at the bifurcation of the smaller vessels into the capillaries extending therefrom.

FIG. 4 is a schematic view of particles of two different sizes, in which a Therapeutic is contained, which are lodged at the bifurcations of both a blood vessel and the smaller vessels extending therefrom.

FIG. 5 is a schematic view of particles containing a Therapeutic, lodged in two smaller vessels, whose degradation or dissolution times are different, showing one partially degraded and one still intact.

FIG. 6 is a schematic view of particles containing a Therapeutic, in which the particles are of two different sizes, the larger of which is designed to degrade or dissolve faster than the smaller particles or vesicles, showing the larger particle partially degraded in a blood vessel, while those lodged in the smaller vessels are still intact.

FIG. 7 is a schematic view of a relatively larger particle containing a Therapeutic, in which the larger particle is almost entirely degraded or dissolved, allowing the Therapeutic to enter the smaller vessels, which remain blocked by intact, smaller, slower degrading or dissolving particles.

FIG. 8 is a schematic view of particles containing a Therapeutic, which are designed to degrade or dissolve at three different rates, showing an almost completely degraded or dissolved relatively larger particle at the bifurcation of a blood vessel, with the Therapeutic being released into the smaller vessels extending therefrom, wherein one of the relatively smaller particles has partially degraded or dissolved, while the other, relatively slower degrading or dissolving smaller particle remains intact, both of which are blocking blood flow into the capillaries.

FIG. 9 is a schematic drawing depicting the trapping of hydrophilic Therapeutics in the aqueous core and in the aqueous layers between the lipid multi-lamellar layers of a liposome, with lipophilic Therapeutics trapped in the lipid multi-lamellar layers thereof.

### DESCRIPTION OF THE PREFERRED EMBODIMENTS

Physiologically compatible materials, which rapidly degrade or dissolve in contact with blood, interstitial fluids or tissue, can be used to create particles or vesicles, containing a Therapeutic, with a diameter of 7 microns or larger. Such materials include liposomes, microspheres, excipients commonly used in pharmaceuticals and other compounds and compositions, as known in the art.

A Therapeutic can be incorporated in particles of two or more sizes. The particles can also be designed to degrade, decompose or dissolve at different times. Employing either of these techniques, preferably employing both of these techniques, reduces the risk of an excessive number of infarcts from occurring in blood vessels of the target organ or tissue at one time. Infarcts in an excessive number of arteries occurring at one time can cause an acute myocardial infarction in the heart, a stroke in the brain, an embolism in the lung and the death of tissue in other organs.

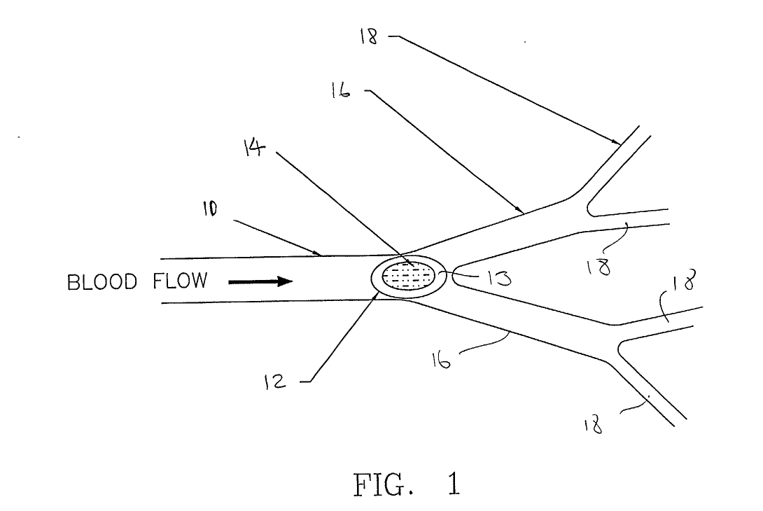

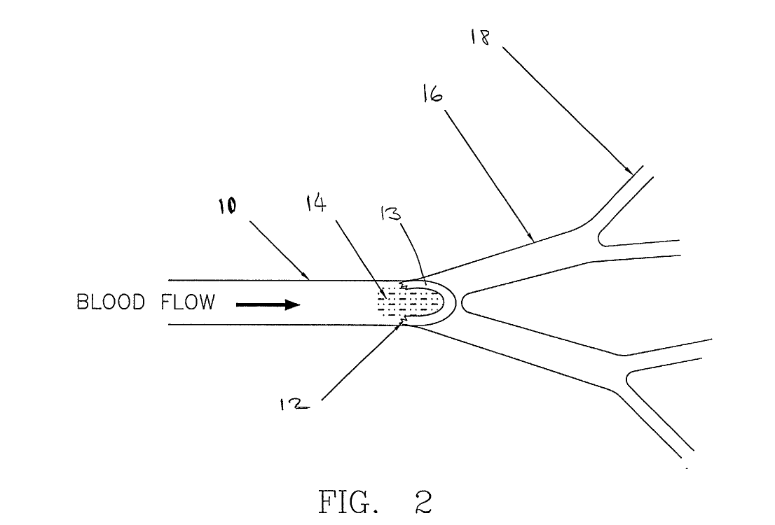

Referring to the drawings, FIG. 1 illustrates a physiologically compatible particle 12 containing a Therapeutic 14, enveloped by encapsulating outer layer 13, lodged in blood vessel 10 at a branch region or bifurcation where relatively smaller blood vessel 16 and capillary 18 extend from blood vessel 10. FIG. 2 illustrates the same blood vessel at a later point in time when encapsulating layer 13 of particle 12 has been partially dissolved or disrupted and Therapeutic 14 is being released in the bloodstream.

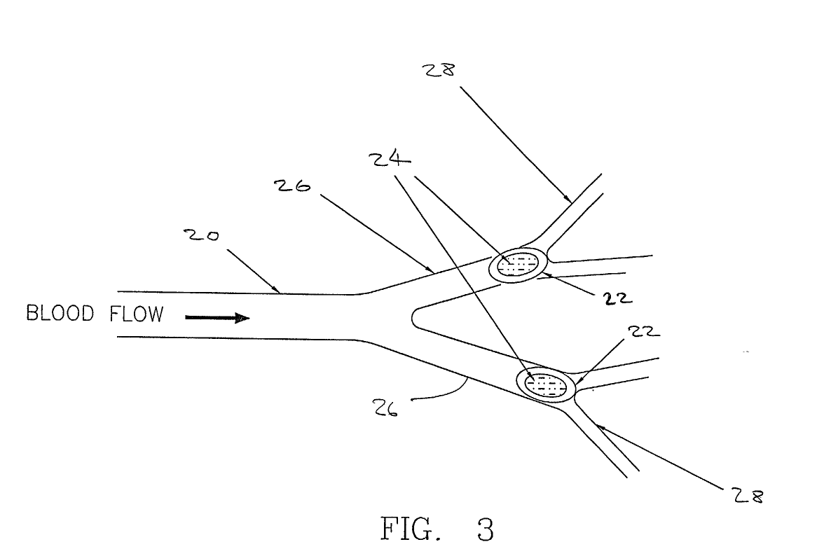

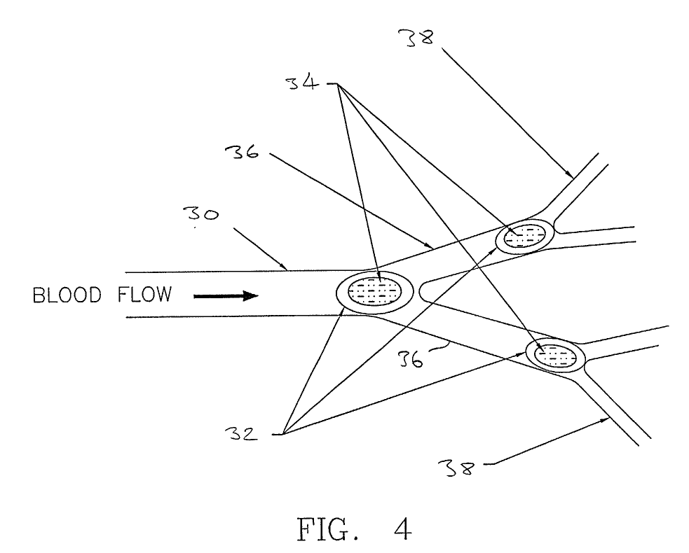

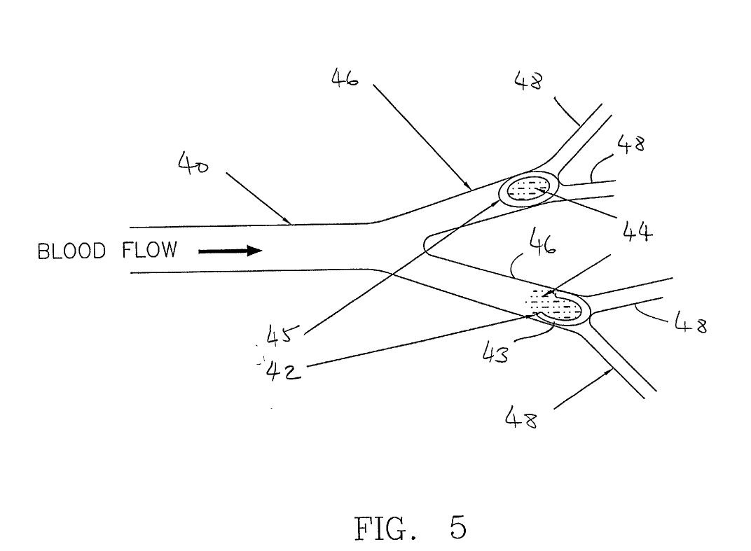

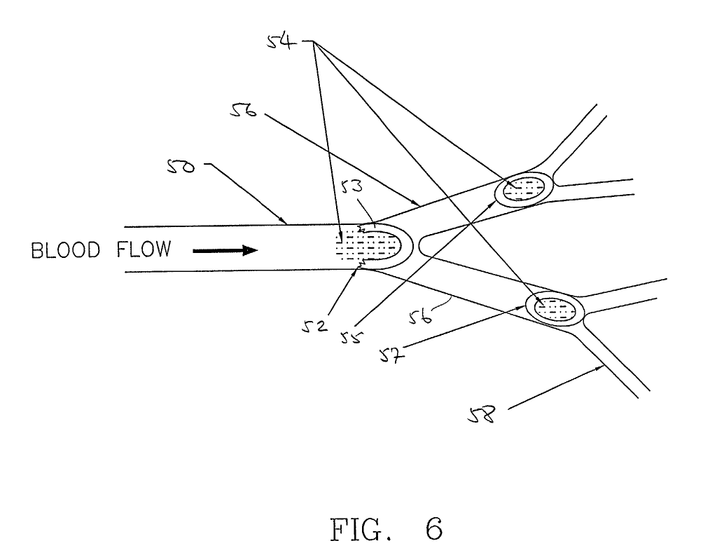

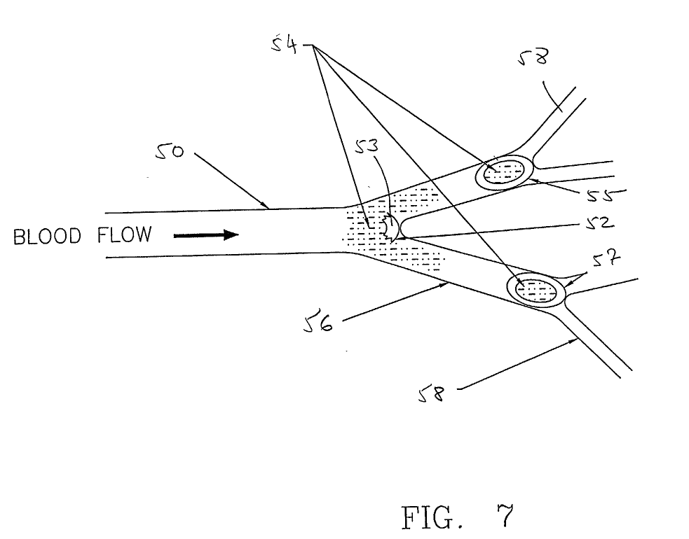

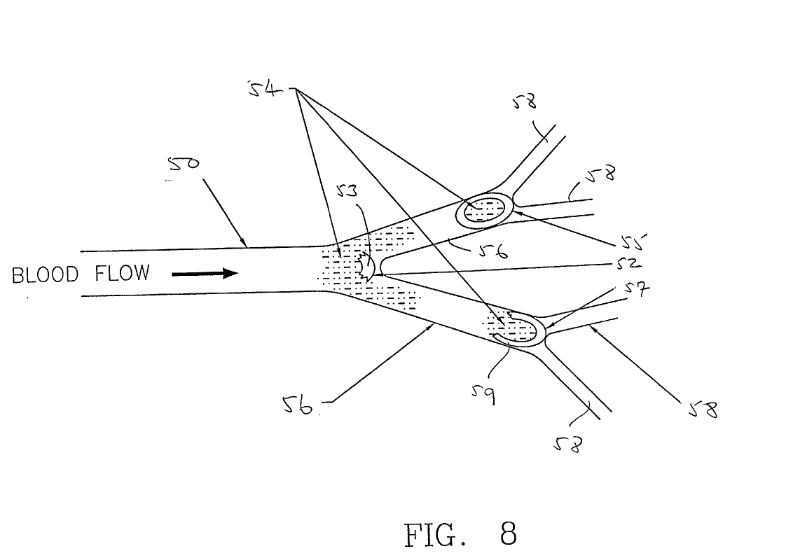

FIG. 3 illustrates blood vessel 20 having relatively smaller blood vessels 26 that branch into capillaries 28 with particles 22 bearing a Therapeutic 24 lodged between the smaller blood vessels 26 and capillaries 28. FIG. 4 illustrates blood vessel 30 having particles 32 bearing Therapeutic 34 lodged at branch regions between blood vessels 30, 36 and 38. FIG. 5 illustrates particles 42 and 45 lodged between blood vessels 46 and 48, and temporarily obstructing blood flow in blood vessel 40. Envelope or outer layer 43 is shown partially disrupted, and some of Therapeutic 44 is being released into blood vessel 46. FIG. 6 shows physiologically compatible particles 52, 55 and 57 trapped in blood vessels 50 and 56, respectively. Encapsulating layer 53 of particle 52 is partially dissolved, and some of the Therapeutic 54 contained within particle 52 is being released into blood vessel 50. FIG. 7 shows blood vessel 50 at a later point in time when layer 53 of particle 52 is almost fully dissolved, and Therapeutic 54 is released not only in blood vessel 50 but also in the relatively smaller blood vessels 56 while capillaries 58 remain blocked by particles 55 and 57. FIG. 8 shows blood vessels 50, 56 and 58 at a still later point in time when layer 59 of particle 57 is partially dissolved, and Therapeutic 54 is being released also from particle 57.

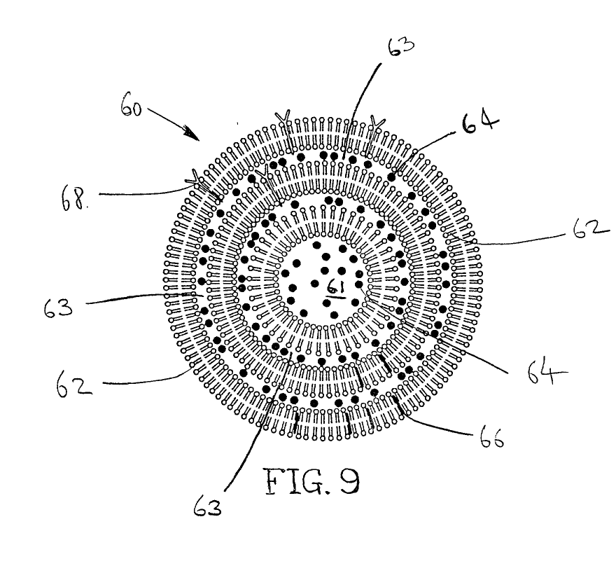

FIG. 9 schematically illustrates a liposome 60 which includes an aqueous core 61, surrounded by bilipid multilammellar layers 62 which entrap aqueous layers 63. Hydrophilic Therapeutics 64 are trapped in aqueous core 61 and aqueous layers 62. Lipophilic Therapeutics 66 are trapped in bilipid multilammellar layers 62. Therapeutic 68 within the outer lamella of multilammellar layers 62 of liposome 60 may have a lipophilic portion as well as a hydrophilic portion.

Aqueous suspensions of phospholipids and methods of making the same are described by Bangham, A. D. et. al., “The Action of Steroids and Streptolysins on the Permeability of Phospholipid Structures to Cations,” J. Mol. Biol. 13:253-259 (1965).

Gregory Gregoriadis, Drug Carriers in Biology and Medicine, Chapter 14, pages 288341, Academic Press, 1979, describes liposomes on pp. 288-297 as follows:

“Confrontation of water-insoluble polar lipids (e.g. phospholipids) with excess water gives rise to highly ordered assemblages which because of their association with unfavorable entropy are eventually arranged in a system of concentric closed membranes (liposomes). Each of these membranes represents an unbroken bimolecular sheet of lipid molecules separated from neighboring membranes by water. Depending on the procedure for their preparation, liposomes consist of one or more lipid lamellae. Unilamellar liposomes have a minimum size of about 25 nm (diameter) but with the multi lamellar version the diameter can increase to several μm.

“Although phospholipids by themselves are sufficient for the fonnation of liposomes, some of the properties of the latter can be improved upon by the incorporation into the liposomal structure of other lipid soluble compounds. Thus, the stability of the phospholipid bilayers (in terms of both rigidity and permeability) can be altered by the inclusion of a sterol, and the incorporation of a charged amphiphile can not only render the liposomal surface positively or negatively charged, it can also increase the distance, and hence aqueous volume, between the bilayers. In the process of their formation liposomes can entrap water solutes in the aqueous channels and subsequently release them at variable rates, This suggests completely closed and selectively permeable phosphollnibilayers (Bangham et al., 1974). Because of such properties, liposomes have served extensively as model membranes to provide information on the inter-relation of membrane lipid components in the basic bimolecular leaflet structure. Liposomes incorporating protein components of membranes (reconstituted liposomes) have been used in the study of lipidprotein interaction in biological membranes, of the action of membrane active compounds such as ionophores, anaesthetics an divalent cations and of the mechanism of antibody-antigen interaction. The liposome drug-carried concept with potential application in biology and medicine is a recent development initiated by the demonstration that liposomes can introduce enzymes into cells and alter their metabolism (Gregoriadis, 1976a, b) and it constitutes the topic of this chapter”.

“A wide variety of substances can be entrapped in liposomes (for more details on methodology see Gregoriadis, 1976c and Tyrrell et al., 1976b). “Entrapment” is taken here to signify either the incorporation of a lipophilic substance into the lipid framework of the bilayer or the passive encapsulation of a water-soluble substance in the aqueous compartments of the system. Macromolecules with both hydrophobic and hydrophilic regions can occupy accordingly the lipid and the aqueous phase of liposomes”.

“The classical procedure for the preparation of liposomes entails dissolution of appropriate concentrations of phospholipids in an organic solvent, its evaporation and the subsequent disruption of the dry lipid layer with excess water or buffer. This leads to the spontaneous formation of multi lamellar liposomes of heterogeneous size. For entrapment, substances which are soluble in organic solvents can be mixed with the lipids in the initial stage of the procedure thus ending up as constituents of the membrane structure in the final preparation. On the other hand, water-soluble substances added into the solution used in the dry lipid disruption step will end up in the aqueous channels. It is conceivable that some macromolecules may be partially submerged into the lipid lamellae. Subsequent sonication of multi lamellar liposomes will lead to the formation of smaller structures, many of them unilainellar. Isolation of such small vesicles from contaminating large ones can be effected by molecular sieve chromatography (Huang, 1969) or by ultracentrifugation (Barenholtz et al., 1977). Separation of liposomes with entrapped materials from the unentrapped solutes can be carried out by various methods depending on the physical characteristics (e.g. size, charge, etc.) of both liposomes and solutes. For instance, when dealing with small molecular weight substances, elimination of unentrapped material by dialysis is preferable since there is neither loss of liposomes nor large increase of the final volume of the preparation, both occurring with other methods. Centrifugation can be applied with preparations containing large liposomes, although by this method it is possible to lose in the supernatant some of the smaller structures. With liposomes below certain size, unentrapped material can be eliminated by molecular sieve chromatography. However, some of the preparations applied may be partially absorbed on the column. Furthermore, the final volume of the liposomes received, usually at the end of the void volume, will be increased considerably”.

“Preparation of liposomes by sonication has certain disadvantages. For instance, in spite of precautions, unsaturated bonds in the fatty acid chains of phospholipids can be oxidized forming lyso-compounds and free fatty acids. In addition, some substances destined for entrapment (e.g. certain proteins, nucleic acids, etc.) are prone to denaturation or otherwise inactivation. However, a method (Batzri and Kom, 1975) has been devised for preparing liposomes of the same size as that of sonicated liposomes by injecting an organic solution of the lipids into a large aqueous volume (containing the substance for entrapment). Although sonication is avoided, entrapment is rather low and isolation of the liposomes formed is tedious. Both methods of sonication and injection give rise to vesicles which because of their small internal aqueous space cannot accommodate particulate mater such as viruses and DNA fragments or even some large macromolecules. Techniques have been developed for the preparation of large unilamellar or oligolamellar liposomes (μm dimension) in the presence (Deamer and Bangham, 1976) or absence (Papahadjopoulos et al., 1975) or organic solvents. With both methods, however, entrapment is low. This problem appears to have been eliminated in a recent technique (Szoka and Papahadjopoulos, 1978) which produces unilamellar or oligolarnellar liposomes with large internal aqueous space and high entrapment yield. Yet in another technique, liposomes are formed from phospholipids mixed with sodium chelate which is subsequently eliminated by gel filtration (Brunner et al., 1976) or by dialysis (Milsmann et al., 1978). More recently, we have found (Wreschner and Gregoriadis, 1978) that upon mixing of negatively and positively charged small liposomes, there is a rapid increase in turbidity due to a aggregation. This is followed by clearance and the formation, apparently through fusion (Wreschner et al., 1978), of hybrid vesicles with properties different from those of the parent vesicles (see legend to FIG. 2 for comments on size). In this way, a substance entrapped in the parent negatively charged population of liposomes can be transferred into the hybrid species without apparent leakage. Hybrid liposomes can be subsequently transformed into large mono or oligolamellar liposomes (FIG. 2). Finally, there has been a recent report on the preparation of small (50-60 nm diameter) liposomes by sonicating diluted samples of rat and mouse plasma (Dunnick and Kriss, 1977). The resulting vesicles, containing roughly equal amounts of plasma lipid and plasma protein, could be made to entrap ^{99m}TcO₄, to which the vesicles were impermeable”.

On page 309 and 310, Gregoriadis also observes, “With multi-lamellar liposomes, after fusion of the outer bilayer with the cell's membrane, the remaining core will end up in the cell where it may be autophagocytosed or fuse with other organelles”, and he notes, “In general, the fate of liposomes injected intravenously is dependent on their physical characteristics (e.g. size, charge, lipid composition, etc.). As mentioned already, liposomes, upon contact with blood, are coated with one or more plasma proteins which alter the net surface charge and electrophoretic mobility of the carrier. On the other hand, when liposomes without (Aborowski et al., 1977) or with only 10% cholesterol (in moles) (Scherphof et al., 1978) are exposed to serum in vitro, a considerable proportion of the entrapped material is liberated”.

“Size and surface charge appear to control the rate of liposome elimination from the blood. Large liposomes are removed more rapidly than small ones (Juliano and Stamp, 1975; Hinkle et al., 1978). Since elimination is the result of uptake by tissues, it is probable that Kupffer cells attract large liposomes rapidly, while the smaller ones (<100 nm) reach the hepatic parenchymal cells at a slower rate. Elimination from the blood is more rapid for negatively charged than for neutral or positive liposomes (Gregoriadis and Neerunjun, 1974)”.

On page 333, Gregoriadis proposes, “In terms of carrier changes, we have proposed (Gregoriadis, 1974) coating of its surface with molecules with a specific affinity for the target. It is expected that such molecules will, by attaching themselves onto the relevant receptors, mediate association of the liposomal moiety (and its drug contents) with the target”.

If the Therapeutic, for example, is physiologically compatible bone marrow, the bone marrow may be passed through screens or sieves and vortexed to create particles or vesicles with a diameter of 7 microns or larger, by means known in the art. The diameter of the bone marrow particles or stem cells occurring therein can be further increased by incorporating them in liposomes, as known in the art. Sonication of bone marrow, however, is not desirable, as the stem cells therein could be damaged or destroyed.

Liposomes can be produced, for example, as described in “Procedure for preparation of liposomes with large internal aqueous space and high capture reverse-phase evaporation” by Szoka, F. et al., Proc. Nat. Acad. Sci, U.S.A., 1978, 75:4194-4198.

In Methods in Enzymology, Volume 149, Chapter 16, titled “Liposomes as Carriers of in Vivo Gene Transfer and Expression”, pg. 157-177, Academic Press, 1987, Nicolau, C., et al., describe in vitro and in vivo gene transfer in eukaryotic cells using liposomes. On page 159, they state, “The encapsulation efficiency is little affected by the DNA concentration, but depends to a considerable degree on the phospholipid concentration. The higher the lipid concentration at a given DNA concentration, the lower the encapsulated aqueous volume”. However, on page 160 they observe, “We find that even 15 sec of mild sonication (10 kHz) degrades most of the DNA (MW>500,000), whereas vortexing even for 8 min is without influence”. On page 168 they note that in both the endothelial cells and the hepatocytes, when lactosylceramide (LacCer) containing liposomes are used a considerably larger amount of the plasmid is found in these cells than with phospholipid (PL) liposomes (FIGS. 3 and 4). This effect is most marked in the endothelial cells.

On page 175, they note that LacCer containing liposomes have an average diameter of 260 mn (0.26 microns). When Nicolau, C. et al. refer to large liposomes, they refer to liposomes up to 2 microns in diameter, too small to be trapped in small arteries and capillaries of organs or tissues as proposed herein.

In “Lipofection: A highly efficient, lipid-mediated DNA-transfection procedure”, Proc. Natl. Acad. Sci., U.S.A. 84: 7413-7417, Felgner, P L, et. al, on page 7413 state, “Due in part to the size and charge of DNA and to the multitude of enzymatic and membrane barriers imposed by the cell, the spontaneous entry of intact DNA into the cell and its subsequent expression in the nucleus is a very inefficient process”.

DOTMA, a dioleyloxy trimethylammonium, is marketed by Life Technologies, Inc. as Lipofecten®, for producing positively charged liposomes. The net positive charge of the DOTMA liposomes enables them to attach to (fuse with) negatively charged lipid membranes and cell surfaces. Also, in “Cationic Liposome-mediated transfection”, Felgner, P L, et al., Nature 337:387-388 (Jan. 26, 1989), the use of DNA containing liposomes to transfect tissues is described.

Hart, S. L., et. al, in “Lipid-Mediated Enhancement of Transfection by a Nonviral Integrin Targeting Vector, Human Gene Therapy”, 9:575-585, Mar. 1, 1998, report that Lipofectin® encapsulated integrin-targeted peptide/DNA avoids endosomal degradation and enhances transfection.

Kim, J. S., et al., in “A new non-viral DNA delivery vector: the terplex system”, Journal of Controlled Release 53:175-182 (1998), report that the toxicity of Lipofecting can be avoided by encapsulating DNA in a liposome consisting of stearyl-poly (L-lysine) and a low density lipoprotein.

Further, Choi, Y. H., et al., in “Polyethylene glycol-grafted pol-Llysine as polymeric gene carrier”, Journal of Controlled Release 54:39-48(1998), report that polyethylene-glycol (PEG) modified liposomes exhibited less toxicity than Lipofectin® with almost equal transfection efficiency.

Floch, V., et al., in “New Biocompatible Cationic Amphiphiles Derivative from Glycine Betaine: A Novel Family of Efficient Nonviral Gene Transfer Agents”, Biochemical and Reophysical Research Communications 251:360-365 (1998), describe glycine betaines as cationic lipid vectors, which produce superior transfection efficiency with less toxicity than Lipofecting in serum, with high biodegradability.

In “A New Efficient Method for Transfection of Neonatal Cardiomyocytes using Histone H1 in Combination with DOSPER Liposomal Transfection Reagent”, Somatic Cell and Molecular Genetics 24(4):257-261 (1998), Kott, M., et al., show that the polycationic lipid, DOSPER, with Histone H1 was more efficient in transfecting neonatal cardiomyocytes than and other cationic lipids.

In all of the above publications, the particle or vesicle diameter was not larger than 2 microns and in most cases was substantially less than 1 micron (typically 50-500 nm or 0.05-0.5 microns) in diameter, which would quickly and easily pass through all blood vessels and capillaries of a tissue or organ and enter the general circulation.

Giant lipid vesicles (liposomes) with diameters of 15 to 56 microns, as described by D. Needham and E. Evans in “Structure and Mechanical Properties of Giant Lipid (DMPC) Vesicle Bilayers from 20° below to 10° C. above the Liquid Crystal-Crystalline Phase Transition at 24° C.”, Biochemistry 27:8261-8269 (1988), may be made by first drying the lipid from chloroform-methanol solvent (as first described by Reeves, J. P. and Dowben, R. M. in J. Cell Biology 73:49-60 (1969) on a Teflon disk roughened with emery paper to form tiny grooves in one direction (rather than the glass plate described by Reeves and Dowben). The disk is preheated to about 400° C. prior to application of 50 μL of 10 mg/ml dimyristoylphosphatidylcholine (DMPC, Avanti Pol Lipids, Alabaster, Aa.) in chloroform-methanol (2:1). The solvent quickly evaporates and the disk with a thin film of lipid is evacuated overnight to assure complete elimination of solvent. The Teflon disk and lipid film are prehydrated at 40° C. for 3 minutes with water-saturated argon. Final hydration is performed by the addition of distilled water at 30-35° C. Strings of vesicles appear which form a cloud in the suspension, are harvested by pipette into a 1 mL Eppendorf tube and diluted in 35° C. water.

Such vesicles are stable for twelve hours or longer at 20° C. and for somewhat shorter periods of time at higher temperatures (up to one hour or longer at 37° C.), and are thus too long-lasting.

In “Electro-mechanical permeabilization of lipid vesicles”, Biophysical Journal 55:1001-1009 (May 1989), Needham, D. and Hochmuth, R. M., describe other lipid materials for creating giant liposomes with diameters of 20 to 40 microns, stearolyleoylphosphatidycholine (SOPC) and dioleoylphosphatidylglycerol (DOPG), with or without cholesterol at about 33 mol %. The addition of negatively charged DOPC, 5 mol %, allows electrolyte suspensions to be obtained. The vesicles are formed by rehydration in a solution of mM sucrose containing 10 mM sodium chloride (NaCl). The lipid cloud is then further diluted with 200 mM glucose, 10 mM NaCl and 0.2% albumin. Such liposomes are stable for up to one hour or longer at 37° C., and are thus too long-lasting.

Zhelev, D. V. and Needham, D., in “Tension-stabilized pores in giant vesicles: determination of pore size and pore line tension”, Biochimica et Biophysical Acta 1147:89-104 (1993), describe giant vesicles with diameters of 25 to 56 microns made with SOPC, with or without 50 mol % cholesterol. Some of the liposomes were single-walled and some were multilamellor. They determined that one or more pores form in the liposomes as the membranes degrade, based on membrane tension, and the pores can be made to close by adjusting the membrane tension. In vivo, however, adjustment of membrane tension is difficult, if not impossible to control.

When injected into a mammalian tissue or blood vessel, liposomes typically degrade by contact with the enzyme, lipase, which degradation generally takes hours to occur, and by the action of macrophages, through the body's reticuloendothelial system, which generally takes hours to days.

Giant liposomes, prepared as described by D. Needham and E. Evans as mentioned above, can be made to degrade, when in contact with tissue or blood, and release the Therapeutic contained therein in less then 3 minutes by two novel means.

In one embodiment, osmolality can be employed to accomplish the desired result. Liposomes can be created in a high salt solution, up to a I molar concentration, about ten times higher than the salinity of blood (approximately 100 millimolar concentration), preferably about three to seven times higher than the salinity of blood. When introduced into tissue or a blood vessel, water flows into the liposomes in an attempt to equalize the osmilality by bringing the liposome's salinity down to the blood's salinity level, causing the liposomes to bloat and then burst, releasing the Therapeutic. However, some Therapeutics may be adversely affected by exposure to a high salt solution.

In a preferred embodiment, giant liposomes can be constructed by including in the liposome at least one component that is unstable at body temperatures, such as a custom acyl composition. Such a liposome can be composed of dioleoylphosphatidylethanolamine (DOPE), dipalmitoylphosphatidylcholine (DPPC) and cholesterol in a ratio of 7:3:5, respectively. Such liposomes are stable at room temperature, about 23° or 24° C. However, when injected into tissue or a blood vessel, due to the instability of DOPE at 35° C. or above, the liposome goes into a hexagonal phase as its temperature reaches about 35° C., causing a rearrangement of the liposome's layers, collapse of the liposome and release of the Therapeutic contained therein. Preparation of such liposomes is known in the art and is described in Yatvin et al., Science 202:1290-1293 (1978).

Other methods may be employed to create large diameter liposomes, greater than 10 microns in diameter, in which a Therapeutic can be incorporated, which degrade and release the Therapeutic when in contact with tissue or blood within 30 seconds to less than 3 minutes.

The time of degradation of a thermally degradable liposome depends upon the ratio of the volume of the injected liposome suspension to the volume of body fluid (blood) in which the liposome suspension is diluted, or the surface area of tissue with which the liposome suspension comes into contact. In physiologically desirable administration volumes, the decomposition time is less than three minutes, typically less than two minutes. If injected into a vessel, the liposomes in the outer area of the injected bolus will reach the degradation temperature earlier than the liposomes in the center, providing a desirable variation in the release times of the Therapeutic. If infused into tissue, the liposomes in contact with the tissue will reach the degradation temperature before those in the center of the injected pool, creating a similar difference in the release times.

Microspheres are described in detail by Okada, H. et al., Critical Reviews in Therapeutic Drug Cancer Systems 12(1):1-99 (1995). While degradation or dissolution times are reported for various formulations of drug containing microspheres, which range in size from less than 1 micron to 290 microns, in hours, for a few formulations, and in days, weeks and months for the others, they mention that spherical, degradable starch microspheres of 40 micron diameter had a degradation half-life of 15-30 minutes in normal serum (Page 13). No shorter degradation or dissolution times are cited.

Among the materials they describe for preparing microspheres are proteins, polypeptides, polysaccharides, aliphatic polyesters of hydroxy acids, polyanhydrides, poly ortho esters and polyalkyleyanoacrylate. While they do not describe microspheres which rapidly degrade or dissolve in a body fluid such as time periods as short as from 30 seconds to 3 minutes, such microspheres can be formulated and a Therapeutic encapsulated therein, by means known in the art.

U.S. Pat. No. 4,093,709 to Choi et al. describes drug delivery devices manufactured from poly/orthioesters and poly/orthocarbonates. An early paper, in which orthoacids are described, is “Derivatives of Orthoacids” by Crank, G. and Eastwood, F. W., Aust. J. Chem., 1964,17:1392-8.

Such materials, designed to rapidly degrade or dissolve in from 30 seconds to 3 minutes, can be prepared by means known in the art and used to encapsulate a Therapeutic.

U.S. Pat. No. 3,922,339 to Shear, J. L. describes the use of common pharmaceutical excipients to form medicant containing particles. Such materials can be mixed with or used to enrobe a Therapeutic so as to produce particles of 7 microns or larger in diameter and formulated to rapidly degrade or dissolve in from 30 seconds to 3 minutes, preferably within 1 to 2 minutes, by means known in the art.

Banker, G. S. et al., Pharmaceutical Dosage Forms, Chapter 2, Marcel Dekker, Inc. (1990), pp. 61-107, describe the formulation and manufacture of oral dosage forms of drugs (tablets) with excipients commonly used in the pharmaceutical industry, including diluents, binders, fillers, lubricants, disintegrants, colorings, sweeteners, flavors, buffers and absorbents. They note that “disintegration and dissolution alone do not ensure Therapeutic activity”. Attention is paid to a variety of factors, including delivery of the correct amount of drug in the right form, as well as stability, physiomechanical properties (size, form, heal sensitivity, taste, color, odor and appearance), physicochemical properties (solubility, pH compatibility, etc.), dissolution time and absorbability. Selected excipients, formulated to rapidly degrade or dissolve in from 30 seconds to 3 minutes, preferably within 1 to 2 minutes, with a diameter of 7 microns or larger, containing a Therapeutic, can be made by means known in the art.

From the above, it can be seen that many physiologically desirable applications for particles or vesicles with a diameter of 7 microns or larger, which rapidly degrade or dissolve in from 30 seconds to 3 minutes, preferably within 1 to 2 minutes, can be used to deliver a Therapeutic for an enhanced, localized effect in an organ or tissue. While such particles or vesicles can be infused into an artery feeding an organ or a portion thereof, or injected directly into the organ or an area thereof, they may also be applied, for example, to the lungs in an aerosol formulation.

In the practice of this invention, rapidly degrading or dissolving particles or vesicles, containing a Therapeutic such as an angiogenic growth factor, angiogenic gene, physiologically compatible bone marrow or stem cells, with a diameter of 7 microns or larger, could be infused into one or more of the coronary arteries of the heart or injected into the heart muscle to treat ischemia due to coronary artery disease. Peripheral atherosclerosis in the limbs can be similarly treated. In addition, genes, such as those expressing adenylyl cyclase to enhance c-AMP signalling, can be administered in such particles or vesicles to treat cardiomyopathy or congestive heart failure.

Physiologically compatible bone marrow and/or stem cells, contained in rapidly degrading or dissolving particles or vesicles of the size described herein, can be injected into an area of the brain or infused into an artery feeding a portion of the brain to supply cells, myelin or other constituents of brain tissue in which that area is deficient, as well as to provide angiogenic growth factors to revitalize brain tissue as the result of a stroke, injury or disease. Genes encapsulated in such particles can likewise be used to cause the expression of needed substances.

Autologous or embryonic endothelial cells (ECs) and/or endothelial progenitor cells (EPCs) may also be encapsulated or incorporated into liposomes or particles with a diameter of 7 microns or larger, preferably 20 to 60 microns in diameter, which degrade or dissolve in 30 seconds or longer, but less than 3 minutes. ECs and EPCs provide a source of cells which can be mobilized by endogenous angiogenic growth factors, as well as angiogenic growth factors, encased or incorporated with the ECs or EPCs in such liposomes or particles, to create blood vessels or other needed cell types in the organ or tissue. ECs and EPCs can be harvested from bone marrow or peripheral blood and grown in culture ex-vivo, by means known in the art, in order to increase the number available for this purpose.

To treat an infection or inflammation in an organ or tissue, an antibiotic or anti-inflammatory can be incorporated into a particle with a diameter exceeding 7 microns, which is designed to degrade or dissolve in 30 seconds to 3 minutes, and injected into the organ or tissue or a blood vessel supplying the same.

While microspheres and excipient containing particles are typically made very small, to enable them to pass through blood vessels and capillaries, they can be formed with any desired diameter, by means known in the art, and a Therapeutic incorporated therein, to fulfill the purpose of this invention.

Therapeutics to be used in such particles can be solids or liquids, soluble or insoluble, lipophilic as well as hydrophilic or both. Therapeutics can be contained in, entrapped in, enrobed by, attached to, admixed with or otherwise made a part of particles with diameter exceeding 7 microns in diameter and which are formulated to dissolve or degrade in a time period of about 30 seconds to less than 3 minutes.

Many other applications within the spirit and scope of the present invention will become apparent to one skilled in the art. The present invention is not limited by the foregoing illustrative description but by the appended claims.

I claim:

1. A physiologically compatible particle containing at least one therapeutic agent; said particle having a diameter of at least about 7 microns but no more than about 300 microns and a residence time in a body fluid of at least about one half-minute but less than about 3 minutes.

2. The physiologically compatible particle in accordance with claim 1 wherein the particle is a liposome.

3. The physiologically compatible particle in accordance with claim 2 wherein the therapeutic agent is hydrophilic.

4. The physiologically compatible particle in accordance with claim 2 wherein the therapeutic agent is lipophilic.

5. The physiologically compatible particle in accordance with claim 2 wherein the liposome contains a hydrophilic therapeutic agent and a lipophilic therapeutic agent.

6. The physiologically compatible particle in accordance with claim 1 wherein the therapeutic agent is a biologically active substance.

7. The physiologically compatible particle in accordance with claim 6 wherein the biologically active substance is a gene.

8. The physiologically compatible particle in accordance with claim 6 wherein the biologically active substance is a protein.

9. The physiologically compatible particle in accordance with claim 6 wherein the biologically active substance is an enzyme.

10. The physiologically compatible particle in accordance with claim 6 wherein the biologically active substance is a monoclonal antibody.

11. The physiologically compatible particle in accordance with claim 6 wherein the biologically active substance is a cytokine.

12. The physiologically compatible particle in accordance with claim 1 wherein the therapeutic agent is a nutrient.

13. The physiologically compatible particle in accordance with claim 1 wherein the therapeutic agent is a vitamin.

14. The physiologically compatible particle in accordance with claim 1 wherein the therapeutic agent is a chemotherapeutic substance.

15. The physiologically compatible particle in accordance with claim 1 wherein the therapeutic agent is an organic compound.

16. The physiologically compatible particle in accordance with claim 1 wherein the therapeutic agent is an inorganic compound.

17. The physiologically compatible particle in accordance with claim 1 wherein the therapeutic agent is bone marrow.

18. The physiologically compatible particle in accordance with claim 1 wherein the therapeutic agent is a stem cell.

19. The physiologically compatible particle in accordance with claim 1 wherein the therapeutic agent is fibroblast growth factor.

20. The physiologically compatible particle in accordance with claim 1 wherein the therapeutic agent is vascular endothelial growth factor.

21. The physiologically compatible particle in accordance with claim 1 which is a microsphere.

22. A method for introducing a therapeutic agent into the vasculature of a patient which comprises injecting into a patient's blood stream physiologically compatible discrete particles containing at least one therapeutic agent, which particles have a diameter of at least about 7 microns but no more than about 300 microns; said particles having a residence time in the patient's blood stream of at least about one-half minute but less than about 3 minutes.

23. The method in accordance with claim 22 wherein the physiologically compatible discrete particles are liposomes.

24. The method in accordance with claim 22 wherein the physiologically compatible discrete particles are microspheres.

25. The method in accordance with claim 22 wherein the therapeutic agent is hydrophilic.

26. The method in accordance with claim 22 wherein the therapeutic agent is lipophilic.

27. The method in accordance with claim 22 wherein the particles are liposomes that contain a hydrophilic therapeutic agent with a lipophilic therapeutic agent.

28. The method in accordance with claim 22 wherein the therapeutic agent is a biologically active substance.

29. The method in accordance with claim 28 wherein the biologically active substance is a gene.

30. The method in accordance with claim 28 wherein the biologically active substance is a protein.

31. The method in accordance with claim 28 wherein the biologically active substance is an enzyme.

32. The method in accordance with claim 28 wherein the biologically active substance is a monoclonal antibody.

33. The method in accordance with claim 28 wherein the biologically active substance is a cytokine.

34. The method in accordance with claim 22 wherein the therapeutic agent is a nutrient.

35. The method in accordance with claim 22 wherein the therapeutic agent is a vitamin.

36. The method in accordance with claim 22 wherein the therapeutic agent is a chemotherapeutic substance.

37. The method in accordance with claim 22 wherein the therapeutic agent is an organic compound.

38. The method in accordance with claim 22 wherein the therapeutic agent is an inorganic compound.

39. The method in accordance with claim 22 wherein the therapeutic agent is a stem cell.

40. The method in accordance with claim 22 wherein the therapeutic agent is bone marrow.

41. The method in accordance with claim 22 wherein the therapeutic agent is fibroblast growth factor.

42. The method in accordance with claim 22 wherein the therapeutic agent is vascular endothelial growth factor.
